(19) United States
(12) Patent Application Publication
(10) Pub. No.: US 2018/0004903 A1
(43) Pub. Date: Jan. 4, 2018

(54) ANALYSIS SYSTEM AND ANALYSIS METHOD

(71) Applicant: Hitachi, Ltd., Tokyo (JP)

(72) Inventors: Hidekatsu TAKADA, Tokyo (JP); Takanobu OOSAKI, Tokyo (JP); Hideyuki BAN, Tokyo (JP)

(21) Appl. No.: 15/541,831

(22) PCT Filed: May 12, 2015
(86) PCT No.: PCT/JP2015/063609

§ 371 Date: Jul. 6, 2017

(51) Int. Cl. G06F 19/00

(52) U.S. Cl. CPC G06F 19/328; G06F 19/3437

(57) ABSTRACT

An analysis system, which includes a processor and a memory connected with the processor, further includes: a model applying unit that predicts at least one change among changes between the conditions of target persons in the case of an intervention not being followed and the conditions of the target persons in the case of the intervention being followed with reference to the health checkup information, the medical information, and the clinical condition transition models; and a simulation unit that predicts medical care expenses using the conditions predicted by the model applying unit, and calculates the medical care expense of a group to which the target persons belong by aggregating the predicted medical care expenses of the individual target persons. In addition the simulation unit outputs screen data used for displaying the calculated medical care expense of the group.

### TECHNICAL FIELD

The present invention relates to an analysis system for supporting health.

### BACKGROUND ART

Recently, health guidances for preventing people from suffering lifestyle-related diseases or from developing these diseases more seriously have been widely provided. For example, health promotion programs such as weight reduction guidances, diet guidances, and walking events are provided. Program providers such as insurance providers determine the contents of programs to be provided and target persons for the programs and make execution plans before providing health guidances to the target persons.

Japanese Unexamined Patent Application Publication No. 2004-310209 (Patent Literature 1) is disclosed as one of background technologies relating to this technology. Patent Literature 1 is a health management support system that includes: a diagnosis result input unit for inputting the data of health checkup results; a high-risk group selection unit for selecting persons who belong to high-risk groups on the basis of the data of health checkup results as thorough checkup target persons; a special management target person selection unit for selecting persons for whom special managements are necessary among the thorough checkup target persons as special management target persons on the basis of the thorough checkup results of the thorough checkup target persons; and a special treatment target person selection unit for selecting persons who still belong to high-risk groups among the special management target persons as special treatment target persons on the basis of the data of follow-up health checkup results obtained for the special management target persons.

### CITATION LIST

### Patent Literature

Patent Literature 1: Japanese Unexamined Patent Application Publication No. 2004-310209

### SUMMARY OF INVENTION

### Technical Problem

Because resources such as expenses for providing health guidances are limited, it is necessary to make use of available resources effectively. Therefore, a system that supports the effective and efficient operations of health guidances is desired. To achieve such a purpose, it becomes very important to plan and carry out an appropriate health guidance through not only analyzing contemporary situations but also by predicting future situations.

A health guidance is often carried out for a group of insured persons or the like. However, although a health guidance (intervention) for an individual person and the change (effect of the health guidance) of his/her clinical condition can be predicted, an effect that is expected at the planning stage of the health guidance cannot be achieved in some cases if the health guidance is carried out for a group. For example, if the participation rates, the persistence rates, and the level of seriousness of the group members at the health guidance program are lower than those that were expected at the planning stage of the health guidance program, the effect of health improvement that was expected at the planning stage cannot be obtained. Therefore, it is required that, in the case of a group of persons being a target, the effect of a health guidance should be analyzed at the time of making the health guidance plan.

### Solution to Problem

One of typical examples of inventions disclosed in this application is as follows. To put it concretely, the one of typical examples is an analysis system that includes a processor and a memory connected with the processor, and the analysis system is capable of accessing a database that includes: health checkup information including the health checkup results of target persons; medical information including the medical care expenses of the target persons; and clinical condition transition models in which probability dependencies between nodes corresponding to probabilistic variables representing the conditions of the target persons and nodes corresponding to probability variables of factors that change the conditions of the target persons are defined by directed edges or undirected edges. Furthermore, the analysis system includes: a model applying unit in which the processor predicts at least one change among changes between the conditions of the target persons in the case of an intervention not being followed and the conditions of the target persons in the case of the intervention being followed with reference to the health checkup information, the medical information, and the clinical condition transition models; and a simulation unit in which the processor predicts medical care expenses using the conditions predicted by the model applying unit, and calculates the medical care expense of a group to which the target persons belong by aggregating the predicted medical care expenses of the individual target persons. The simulation unit outputs screen data used for displaying the calculated medical care expense of the group.

### Advantageous Effects of Invention

According to one embodiment of the present invention, the advantageous effects of health guidances can be displayed in an easy-to-understand manner. Problems, configurations, and advantageous effects other than those described above will be explicitly shown by explanations about the following embodiment.

### BRIEF DESCRIPTION OF DRAWINGS

FIG. 1 is a diagram showing an example of a configuration of an analysis system of an embodiment according to the present invention.

FIG. 2 is a diagram showing an example of health checkup information of this embodiment.

FIG. 3 is a diagram showing an example of medical information of this embodiment.

FIG. 4 is a diagram showing an example of arrangement information of this embodiment.

FIG. 5 is a diagram showing an example of clinical condition transition model information of this embodiment.

FIG. 6 is a flowchart of intervention editing processing of this embodiment.

FIG. 7 is a diagram showing an example of an intervention editing screen of this embodiment.

FIG. 8 is a flowchart of simulation execution processing of this embodiment.

FIG. 9 is a diagram showing an example of a simulation executing screen of this embodiment.

### DESCRIPTION OF EMBODIMENTS

Hereinafter, an embodiment of the present invention will be explained with reference to the accompanying drawings.

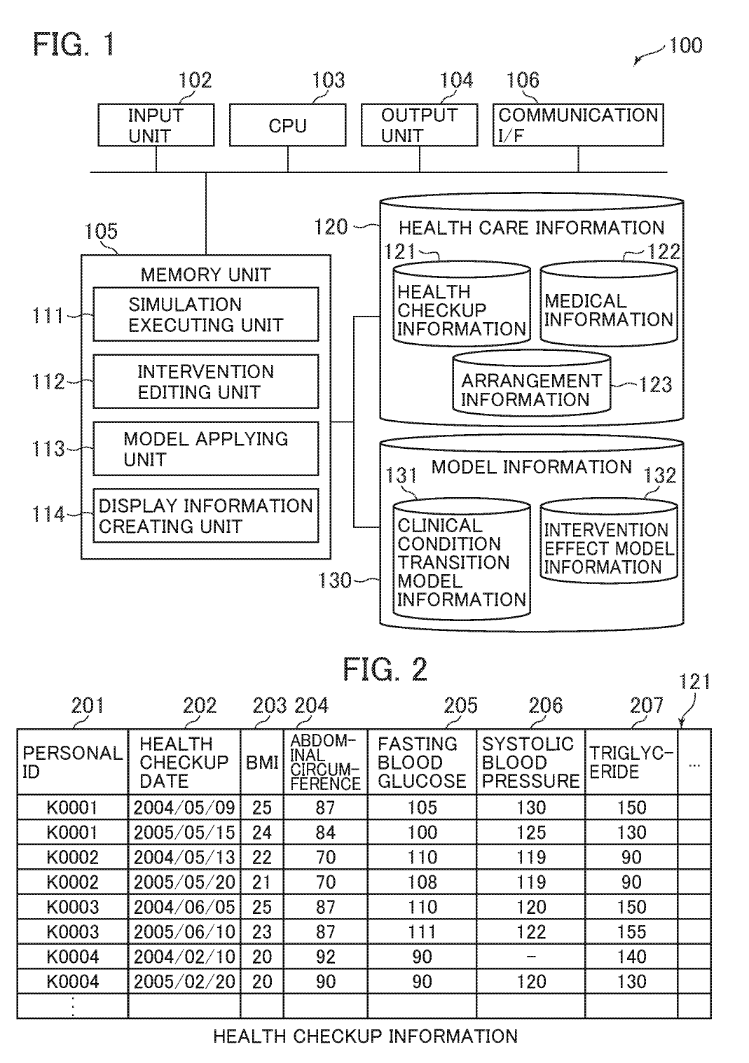

FIG. 1 is a diagram showing an example of a configuration of an analysis system 100 of this embodiment.

The analysis system 100 of this embodiment is a computer that includes an input unit 102, a CPU 103, an output unit 104, a memory unit 105, and a communication interface 106, and analyzes the clinical conditions and medical care expenses of persons belonging to a group by applying clinical condition transition model information 131 and intervention effect model information 132 to the health checkup information 121 and medical information 122 of the persons, and aggregates the analyzed medical care expenses to predict the medical care expenses of the group.

The input unit 102 is a user interface (for example, a keyboard or a mouse) that is used for a user to input data and directions into the analysis system 100. The CPU 103 is a processor that executes programs stored in the memory unit 105. The output unit 104 is a user interface (for example, a display or a printer) that is used for providing the execution results of the programs to a user.

The memory unit 105 includes a memory device such as a memory and an auxiliary memory device. To put it concretely, the memory of the memory unit 105 includes a ROM that is a nonvolatile memory device and a RAM that is a volatile memory. The ROM stores unchanged programs (such as BIOS). The RAM is a high-speed and volatile memory device such as a DRAM (Dynamic Random Access Memory), and temporarily stores programs and data used when the programs are executed, in which the programs and the data have been originally stored in the auxiliary memory device. To put it concretely, the memory stores programs that realizes function blocks such as a simulation executing unit 111, an intervention editing unit 112, a model applying unit 113, and a display information creating unit 114.

The auxiliary memory device of the memory unit 105 is, for example, a high-capacity and nonvolatile memory device such as a magnetic memory device (HDD) or a flash memory (SSD). Here, the auxiliary memory device stores programs and data that are used when the CPU 103 executes the programs. In other words, the programs are read out from the auxiliary memory device, loaded in the memory, and executed by the CPU 103.

The programs executed by the CPU 103 are provided to the analysis system 100 via a removable medium (a CD-ROM or a flash memory) or a network, and stored in a nonvolatile memory device that is a non-temporary memory medium. Therefore, it is recommendable for the analysis system 100 to include an interface via which data is read.

The simulation executing unit 111 executes a simulation for predicting the changes of clinical conditions through the model applying unit 113 applying the clinical condition transition model information 131 or intervention effect model information 132 to the health checkup information 121. The intervention editing unit 112 determines target persons on whom intervention programs are to be executed (hereinafter, referred to as the intervention target persons) in accordance with an input condition. In this embodiment, a piece of information about which intervention programs are to be executed on whom is referred to as an intervention plan. The model applying unit 113 predicts the changes of the clinical conditions of the individual intervention target persons in the case where the intervention programs are not executed on the individual intervention target persons by applying the clinical condition transition model information 131 to the health checkup information 121, and predicts the changes of the clinical conditions of the individual intervention target persons in the case where the intervention programs are executed on the individual intervention target persons by applying the intervention effect model information 132 to the health checkup information 121. The display information creating unit 114 creates screen data for displaying simulation results obtained by the simulation executing unit 111.

The communication interface 106 is an interface for controlling communications with other computers via a network or the like.

The analysis system 100 includes a database that stores health care information 120 and model information 130. Here, it is conceivable that the health care information 120 and the model information 130 are stored in an external database which can be accessed by the analysis system 100.

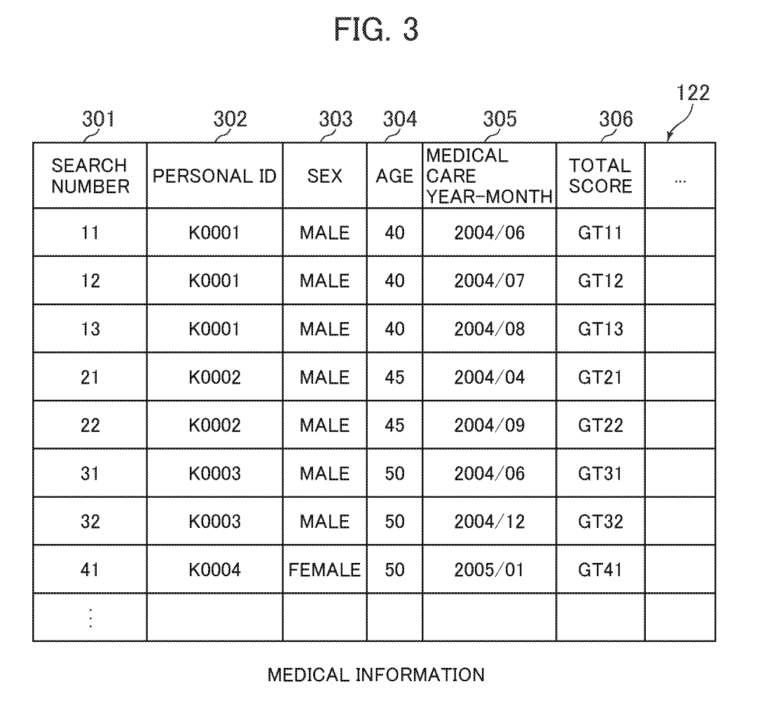

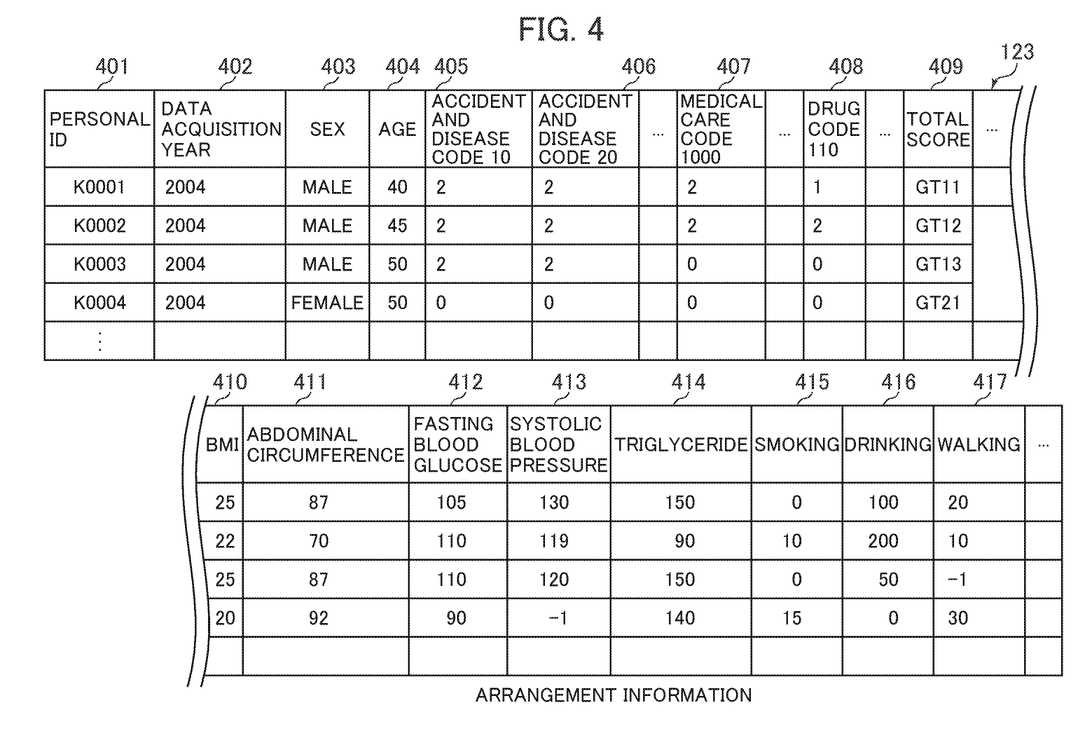

The health care information 120 includes the health checkup information 121 that stores the health checkup results of the individual persons, the medical information 122 that stores information about medical care expenses paid for the medical cares performed on the individual persons by medical institutions, and arrangement information 123 obtained by aggregating the medical information 122. The details of the health checkup information 121, the medical information 122, and the arrangement information 123 will be explained later with reference to FIG. 2, FIG. 3, and FIG. 4 respectively.

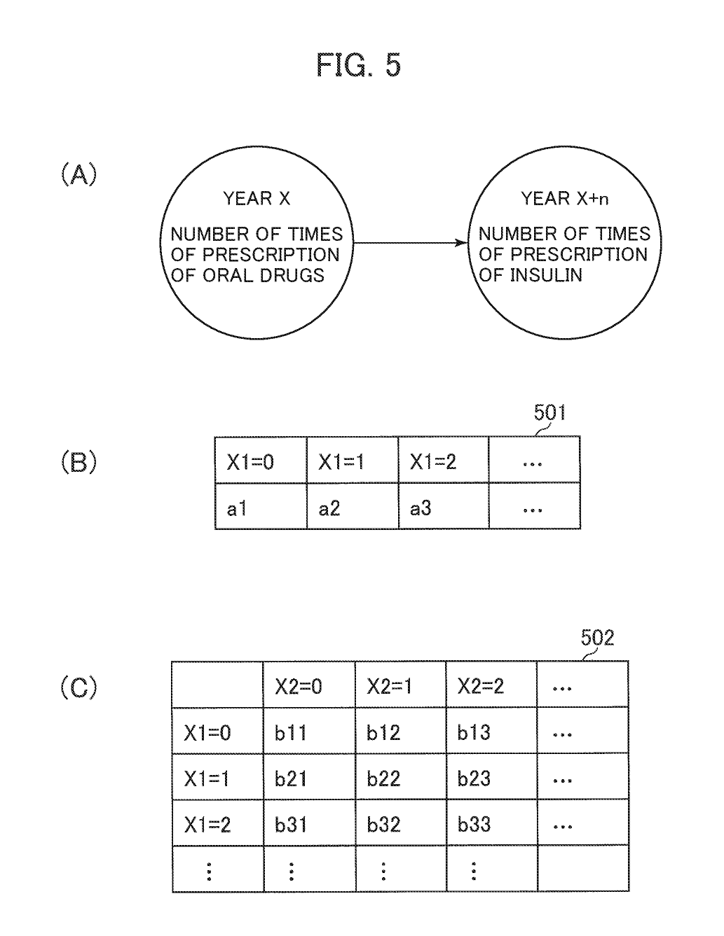

The model information 130 includes the clinical condition transition model information 131 and the intervention effect model information 132. As shown in FIG. 5, the clinical condition transition model information 131 shows a model including: a graph, in which the items of the arrangement information 123 are set as probability variables, the probability variables are set as nodes, and conditional dependences between the probability variables are set as edges; and conditional probability tables. In addition, the intervention effect model information 132 shows a clinical condition transition model in the case where an intervention is performed, and is represented in a similar format as is the case with the clinical condition transition model information 131 shown in FIG. 5, but probability variables are different.

The analysis system 100 of this embodiment is a computer system physically structured on one computer or on plural computers physically or logically combined with each other, and it is conceivable that the analysis system 100 runs using individual threads on the one computer, or runs on a virtual computer built on plural physical computer resources.

FIG. 2 is a diagram showing an example of health checkup information 121 of this embodiment.

The health checkup information 121 includes personal IDs 201 each of which is used for uniquely identifying an individual person, health checkup dates 202, and fields for recording checkup values. A personal ID 201 shows identification information about a person who has a health checkup. A health checkup date 202 is a date when a person has a health checkup. The checkup values include: abdominal circumferences 204 that are the results of abdominal circumference measurements; fasting blood glucose values 205; systolic blood pressures 206; and triglyceride values 207, and the checkup values can include other checkup values as well. Furthermore, the health checkup information 121 can include other kinds of information (for example, lifestyle-related information regarding dietary habit, exercise habit, smoking habit, and the like, and inquiring information).

Here, because a person does not have a specific kind of checkup or for other reasons, there may be case where a part of data of the health checkup information for the person is missed. For example, in FIG. 2, the data of the checkup items that a person with a personal ID “K0004” has in the year 2004 does not include the data of systolic blood pressure 206.

FIG. 3 is a diagram showing an example of medical information 122 of this embodiment.

The medical information 122 is information holding correspondent relationships between receipts and individual persons. The medical information 122 includes search numbers 301, personal IDs 302, genders 303, ages 304, medical care year-months 305, total scores 306, and the like. The search numbers 301 are pieces of identification information each of which is used for uniquely identifying a receipt. The personal IDs 302 are pieces of identification information each of which is used for uniquely identifying a person, and the same identification information as information used for the personal IDs 201 of the health checkup information 121 is used. A gender 303 and an age 304 respectively represent the gender and age of a person. A medical care year-month 305 represents a year-month when the person has a checkup at a medical institution. A total score 306 represents information showing the total score of one receipt.

FIG. 4 is a diagram showing an example of arrangement information 123 of this embodiment.

Each row of the arrangement information 123 shows data aggregated for one year for one personal ID. For example, the arrangement information 123 shown in FIG. 4 includes arranged receipt information obtained by arranging receipt information for the year 2004.

Personal IDs 401, genders 403, and ages 404 are the same as personal IDs 302, genders 303, and ages 304 of the medical information 122 respectively. A data year 402 shows a year in which the relevant arrangement information is created. A total score 409 shows the total sum of medical care expenses used by the relevant person in the relevant year.

An accident and disease code 10 (405) shows the number of receipts with its accident and disease code 10 among the receipts with the relevant personal ID. Similarly, an accident and disease code 20 (406) shows the number of receipts with its accident and disease code 20 among the receipts with the relevant personal ID. A medical care code 1000 (407) shows the number of receipts in the case where medical cares with their medical care code 1000 are provided among the receipts with the relevant personal ID. A drug code 110 (408) shows the number of receipts in the case where drugs with their drug code 110 are prescribed among the receipts with the relevant personal ID.

The arrangement information 123 can include arranged health checkup information obtained by arranging the health checkup information 121. The values of the respective items 410 to 414 of the arranged health checkup information are the values of health checkup data for individual persons and data acquisition years that are shown by the personal IDs 401 and the data acquisition years 402 respectively. This health checkup data can be obtained from the health checkup information 121. If the health checkup information 121 includes plural sets of health checkup data for the same personal ID and for the same year, one of the plural sets of health checkup data for one health checkup date can be used or the average values of the health checkup results of the plural sets for the relevant year can be used. In the case where health checkup data for one health checkup date for each year is used, it is recommendable that data obtained on a general health checkup date which is set in an almost the same season every year is used. Alternatively, it is conceivable that a set of health checkup data that misses less data is selected for each year. Missing data for a checkup item is represented by a predefined value showing that the data for the checkup item is missing. In the example shown in FIG. 4, “−1” was used as the predefined value. Here, all the values for a person who does not have records in the health checkup information 121 are regarded as missing data.

The arrangement information 123 can include arranged inquiring information obtained by arranging the inquiring information. The values of the respective items 415 to 417 of the arranged inquiring information are the values of inquiring data for individual persons and years that are shown by the personal IDs 401 and the data acquisition years 402 respectively. This inquiring data can be obtained from inquiring information (not shown) of the results of inquiries performed at health checkups. If the inquiring information includes plural sets of inquiring data for the same personal ID and for the same year, one of the plural sets of inquiring data for one health checkup date can be used or the average values of the inquiring results of the plural sets for the relevant year can be used. In the case where inquiring data for one health checkup date for each year is used, it is recommendable that data obtained on a general health checkup date which is set in an almost the same season every year is used. Alternatively, it is conceivable that a set of health checkup data that misses less data is selected for each year. Missing data for a checkup item is represented by a predefined value showing that the data for the checkup item is missing. In the example shown in FIG. 4, “−1” is used as the predefined value. Here, all the values for a person who does not have records in the health checkup information are regarded as missing data.

The arrangement information 123 can be created by the analysis system 100 by aggregating medical information 122 as needed, or arrangement information 123 that has already been created from the medical information 122 can be used.

The analysis system 100 of this embodiment calculates the average medical care expense for each disease from the arrangement information 123. To put it concretely, the average of the medical care expenses of persons who suffered from the relevant disease can be set as the average medical care expense.

FIG. 5 is a diagram showing an example of clinical condition transition model information 131 of this embodiment. Here, as described above, the intervention effect model information 132 is represented in the same format as the clinical condition transition model information 131 as shown in FIG. 5.

The clinical condition transition model information 131 includes plural clinical condition transition models. One clinical condition transition model includes: a graph, in which the items of the arrangement information 123 are set as probability variables, the probability variables are set as nodes, and conditional dependences between the probability variables are set as edges; and conditional probability tables. Here, there are two types of edges, that is, one is a directed edge, and the other is an undirected edge. Here, it is defined that a set of nodes is represented by V, and a set of edges is represented by E, and a graph is defined by G=(V, E). A clinical condition transition model is represented by a graphical model such as a Bayesian network or a Markov network.

FIG. 5(A) shows an example of a simple clinical condition transition model including two nodes. “YEAR X NUMBER OF TIMES OF PRESCRIPTION OF ORAL DRUGS” is a probability variable representing the number of times oral drugs are prescribed in the year X, and “YEAR X+n NUMBER OF TIMES OF PRESCRIPTION OF INSULIN” is a probability variable representing the number of times insulin is prescribed in the year X+n. Assuming that the nodes that represent the probability variables are set as v1 and v2 respectively, the graph shown in FIG. 5(A) is comprised of v1, v2 and a directed edge e1 the direction of which is from v1 to v2. If V=(v1, V2) and E=(e1) are defined, the graph shown in FIG. 5(A) can be represented as G=(V, E).

Next, a conditional probability table will be explained as follows. If probability variables represented by node v1 and node v2 are set as x1 and x2 respectively, the graph G in FIG. 5(A) suggests that the joint distribution p(x1, x2) of x1 and x2 is given by p(x1, x2)=p(x2|x1)×p(x1). In other words, the probability distribution of x2 depends on the value of x1, and it is given by a probability p (x2|x1) that is a conditional probability regarding x1. Because the probability variable x1 has no parent node, the probability distribution of x1 becomes p(x1). The conditional probability table includes the values of p(x1) and p(x2|x1). The probability table of p(x1) includes probability values of the respective values of x1. An example of the probability table of p(x1) is shown by a probability table 501 shown in FIG. 5(B). In the table 501, for example, p(x1=0) shows that a probability of x1=0 is a1. This probability can be obtained by calculating the ratio of the number of persons who are not given prescription of oral drugs in the year X to the number of events (the number of persons) included in receipt arrangement information for creating the model. In a similar way, probabilities a2 and a3 can be calculated. Because p(x1) is a probability distribution, Ep(x1)=1. Here, the summation is executed on all the values of x1. The probability table of p(x2|x1) can be obtained by calculating p(x2|x1) for a combination of each value of x1 and each value of x2. For example, p (x2=s2|x1=s1) can be obtained by calculating the ratio of the number of events where x1=s1 and X2=s2 to the number of events where x1=s1. Using the above described calculations, the probability tables can be created.

In such a simple case as is shown in FIG. 5(A) and FIG. 5(B), the graph shown in FIG. 5(A) and the probability table shown in FIG. 5(B) can be regarded as a graphical model. Using this model, it becomes possible to calculate, for example, a probability distribution that insulin will be prescribed for a certain insured person n years later if the number of times oral drugs are prescribed for him/her in a certain year is obtained. For example, if oral drugs are prescribed for a person once this year, a probability that insulin will be prescribed for him/her twice n years later is represented by P(x2=2|x1=1).

The model shown in FIG. 5(A) and FIG. 5(B) is a simple model including only two nodes, but generally speaking, a clinical condition transition model includes edges between plural nodes. For example, a probability table of a clinical condition transition model having n starting nodes (n represents the number of the starting nodes) is represented by n-dimensional table as shown in FIG. 5(C). FIG. 5(C) shows a two-dimensional probability table of a clinical condition transition model having two starting nodes.

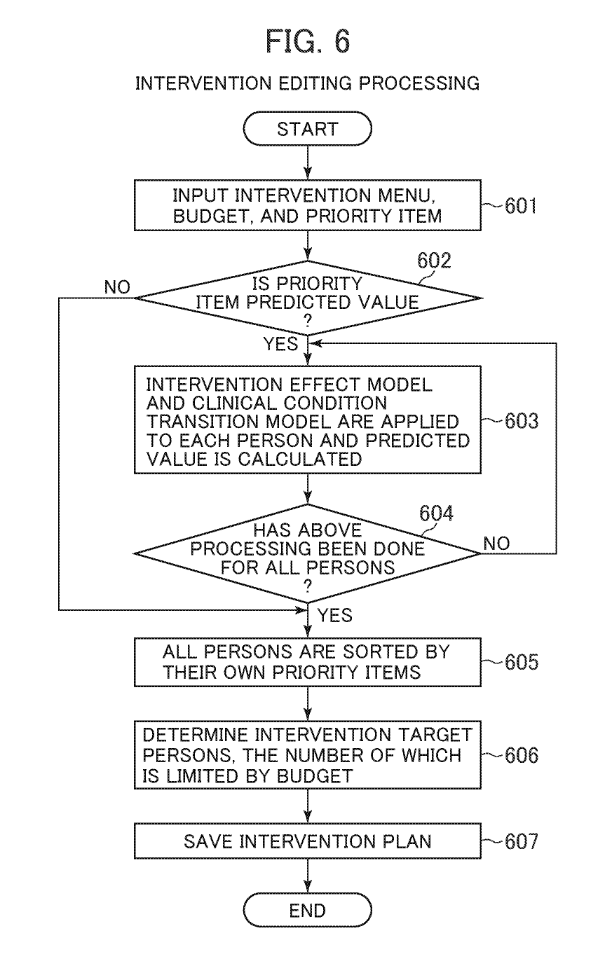

FIG. 6 is a flowchart of intervention editing processing of this embodiment.

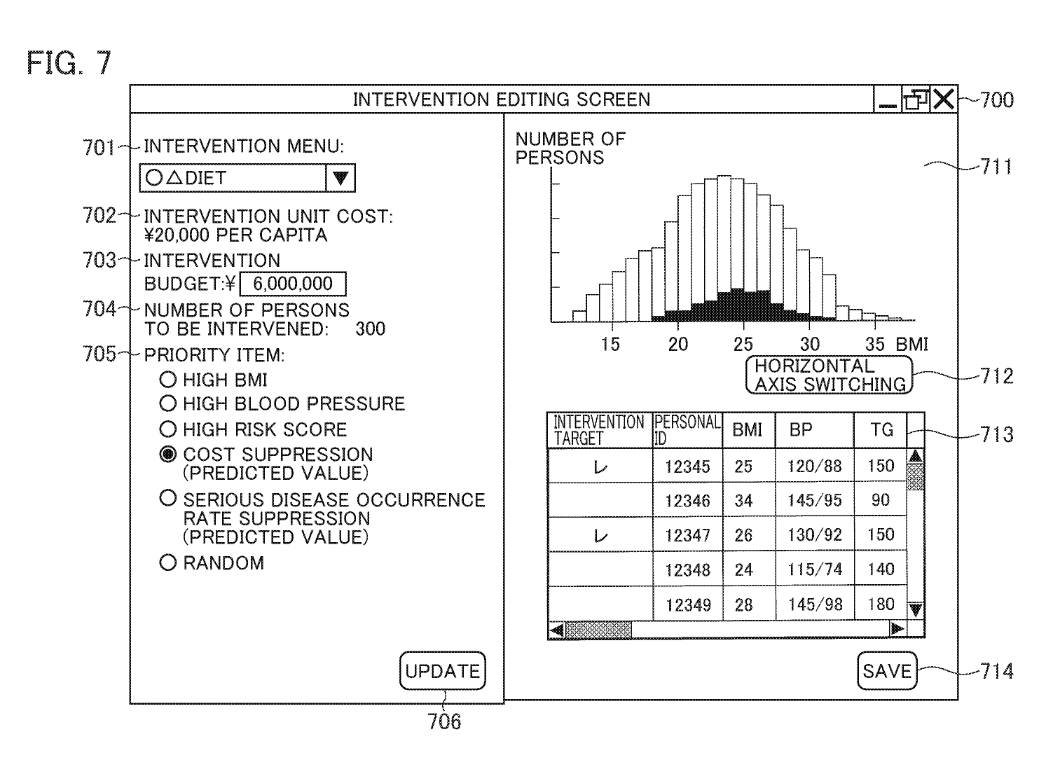

First, the intervention editing unit 112 outputs an intervention editing screen 700 (FIG. 7), and urges a user to input an intervention menu and a condition for creating an intervention plan about who are made intervention target persons for the intervention menu on the basis of a budget, a priority item, and the like (601). At this moment, nothing is displayed in a histogram display area 711 and in a target person list display area 713 on the right side of the intervention editing screen 700. Next, the intervention editing unit 112 judges whether the input priority item is a predicted value or not (602). The priority item is an item that is taken into consideration on a priority base in selecting intervention target persons as shown in FIG. 7, and it is defined by the user of the analysis system 100. Priority items include definite values such as checkup values and predicted values such as future costs. If a priority item selected by the user is a definite value, the flow proceeds to step 605.

On the other hand, if the priority item selected by the user is a predicted value, the intervention editing unit 112 calls up the model applying unit 113, brings out the health checkup results and clinical conditions of the individual persons from the health checkup information 121 and the medical information 122, and calculates a predicted value by applying intervention effect models and clinical condition transition models to the health checkup results and the clinical conditions (603). For example, applying an intervention effect model and a clinical condition model to the health checkup result and clinical condition of an individual person by treating the health checkup result and the clinical condition as known values makes it possible to calculate the onset probabilities of the respective diseases of the individual person after n years. In addition, multiplying the onset probabilities of the respective diseases with the average medical care expenses of the respective diseases and aggregating the obtained products make it possible to calculate the predicted medical care expense of the individual person after n years. If the calculation of the predicted values for all the simulation target persons (to be described later) is finished (YES at step 604), the repeating processing at step 603 is finished, and the flow proceeds to step 605.

Afterward, the intervention editing unit 112 sorts all the persons by the values of their own priority items (605), persons are selected in the order of their rankings from the highest until the number of the selected persons reaches the number defined by the budget, and intervention target persons are determined (606). The intervention editing unit 112 saves the created intervention plan in the memory unit 105 (607).

FIG. 7 is a diagram showing an example of the intervention editing screen 700 output by the analysis system 100 of this embodiment.

An input area for inputting conditions for creating an intervention plan is provided on the left side of the intervention editing screen 700. In this condition input area, an intervention menu input column 701, an intervention budget input column 703, and a priority item selection column 705 are provided.

When the user selects an intervention menu in the intervention menu input column 701, an intervention unit cost 702 set for the intervention menu is displayed. As intervention menus, daily exercises such as a diet menu for losing weight and walking are set in advance. An intervention unit cost is, for example, an expense for getting medical care and/or an initial expense per capita for the intervention menu in one year as shown in the figure. Furthermore, when the user inputs a budget amount in the intervention budget input column 703, the number of persons who can be intervened is calculated using the input budget, and the calculated number of persons 704 to be intervened is displayed.

In addition, a priority item is selected in the priority item selection column 705 by the user. As priority items, there are high BMI (in the order of descending BMI values), high blood pressure (in the order of descending blood pressure values), high risk score (in the order of descending risk scores representing disease occurrence rates), cost suppression (in the order of descending differences (suppression amounts) between medical care expenses predicted in the future in the cases where intervention menus are performed and in the cases where they are not performed), serious disease occurrence rate suppression (in the order of descending differences between serious disease occurrence rates predicted in the future in the cases where intervention menus are performed and in the cases where they are not performed), random (selected randomly), and the like. Among the above priority items, in the case of cost suppression or serious disease occurrence rate suppression, because intervention target persons are determined by predicting events that will occur in the future, the intervention target persons are determined by predicting the clinical conditions of individual persons in the future with the use of intervention effect models and clinical condition transition models (step 604 in FIG. 6).

When “Update” button 706 is operated after a priority item is selected, the flow proceeds to step 602 of the intervention editing processing, and calculation processing for determining the intervention target persons is started.

Afterward, when step 606 of the intervention editing processing is finished, the intervention editing unit 112 displays information about the determined intervention target persons on the right side of the intervention editing screen 700. For this purpose, the histogram display area 711 and the target person list display area 713 are provided on the right side of the intervention editing screen 700. In the histogram display area 711, the distribution of all the simulation target persons and the distribution of the intervention target persons are displayed. In the histogram display area 711, when “Horizontal Axis Switching” button 712 is operated, a subwindow in which an item representing the horizontal axis is selected is displayed, so that a histogram with the horizontal axis representing the selected item can be displayed. An item represented by the horizontal axis can be one of the priority items or can be an item other than any of the priority items.

Here, in an example of the screen shown in FIG. 7, although “Cost Suppression” is selected in the priority item selection column 705, histograms with the horizontal axis representing “BMI” are displayed in the histogram display area 711. Because “Cost Suppression” and “BMI” are different from each other, the histogram of the intervention target persons is widely distributed around the center of the distribution of all the simulation target persons. On the other hand, if a priority item selected in the priority selection column 705 is the same as an item represented by the horizontal axis, the histogram of the intervention target persons is distributed around a part with a higher horizontal value (or a part with a lower horizontal value) of the distribution of all the simulation target persons.

Persons assigned to the intervention target persons is displayed in the target person list display area 713 so that they can be distinguished from the simulation target persons (for example, they are displayed with marks in “Intervention Target” column). Furthermore, the health checkup results and inquiring results of the individual persons can also be displayed in the target person list display area 713.

When “Save” button 714 is operated by the user, a subscreen into which the name of an intervention plan is input is displayed, and the created intervention plan with a name input by the user can be saved in the memory unit 105. Intervention plans saved in the memory unit 105 can be called up using a simulation executing screen 900, and a simulation is executed using a called-up intervention plan.

As described above, the characteristics of a group comprised of intervention target persons determined by an intervention menu, an intervention budget, and a priority item which are input by a user, and the characteristics of all simulation target persons to which the intervention target persons belong can be displayed at the same time in the intervention editing screen 700.

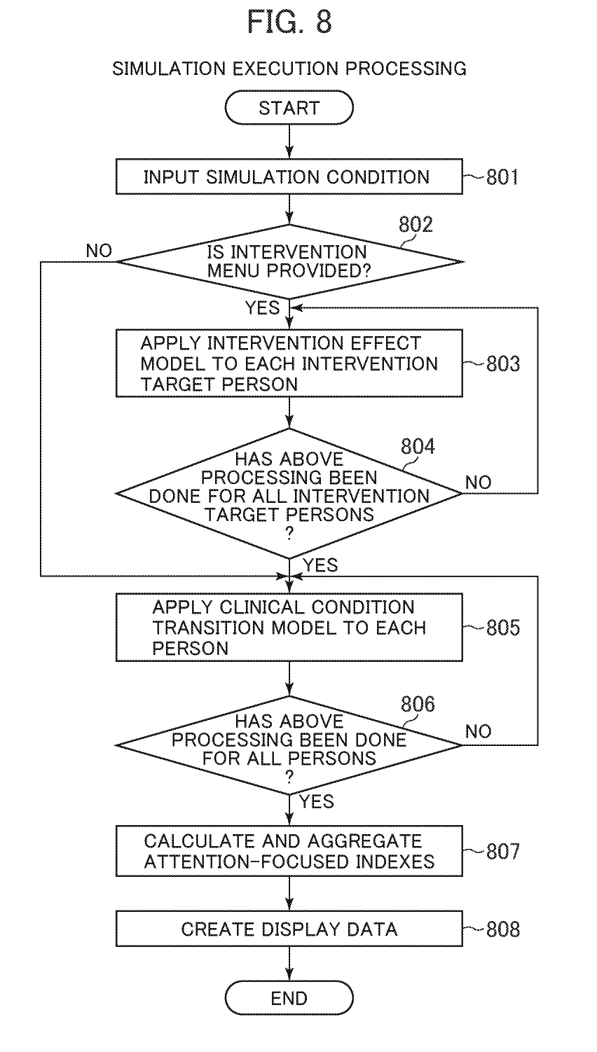

FIG. 8 is a flowchart of simulation execution processing of this embodiment.

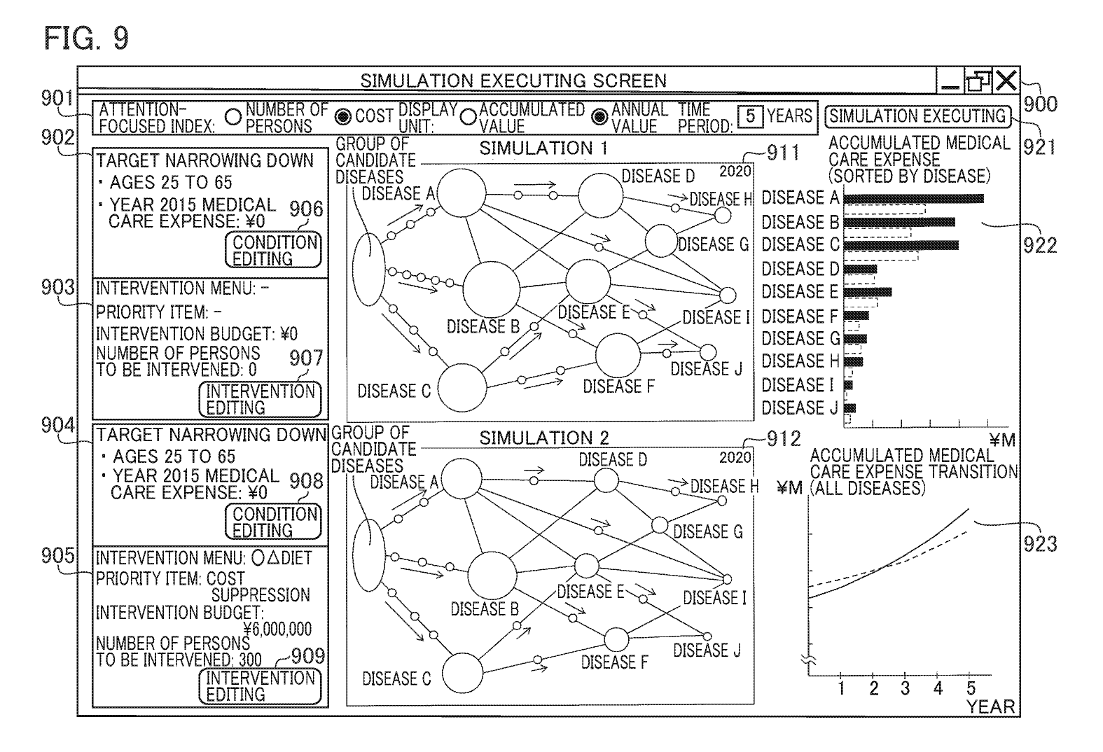

First, the simulation executing unit 111 outputs the simulation executing screen 900 (FIG. 9), and urges a user to input a simulation condition (801). At this moment, nothing is displayed in the simulation result display areas 911, 912, and the accumulated medical care expense display areas 922, 923 of the simulation executing screen 900. As shown in FIG. 9, a user can input plural simulation conditions in order to compare the results of plural simulation results with each other on one screen. Here, even in the case where no intervention menu is performed as a simulation condition (intervention plan) input in step 801, the simulation condition (intervention plan) is treated as one intervention plan.

The simulation executing unit 111 judges whether an intervention menu is provided in the input simulation condition or not (802). As a result, if any intervention menu is not provided, the flow proceeds to step 805.

On the other hand, if an intervention menu is provided, the simulation executing unit 111 calls up the model applying unit 113, applies an intervention effect model with the input simulation condition (intervention plan) to individual intervention target persons, and predicts the clinical condition transitions of the intervention target persons (803). If the prediction of the clinical condition transitions of all the intervention target persons is finished (YES at step 804), the repeating processing at step 803 is finished, and the flow proceeds to step 805.

Afterward, the simulation executing unit 111 predicts the clinical condition transitions of persons whose clinical condition transitions were not predicted at step 803 by applying clinical condition transition models to the individual persons (805). If the prediction of the clinical condition transitions of all the persons is finished (YES at step 806), the repeating processing at step 805 is finished, and the flow proceeds to step 807.

Because the predictions of the clinical condition transitions of individual persons among the intervention target persons and intervention non-target persons are finished through the above processing, the simulation executing unit 111 calculates the attention-focused indexes of the individual persons using the calculated predictions of the clinical condition transitions, and aggregates the attention-focused indexes of the individual persons in a way that the indexes are sorted by the clinical conditions. An attention-focused index is an index set in the simulation executing screen (FIG. 9), and it is “The Number of Persons” or “Cost (Medical Care Expense)”.

Lastly, the simulation executing unit 111 creates data for displaying the aggregated attention-focused indexes, and outputs the created display data (808). It is conceivable that the display data is output to the output unit (display) 104 of the analysis system 100 or to other computers (terminal apparatuses) via the communication interface 106.

FIG. 9 is a diagram showing an example of a simulation executing screen 900 of the analysis system 100 of this embodiment.

The simulation executing screen 900 includes: a display condition setting area 901; target narrowing-down condition setting areas 902 and 904; intervention plan setting areas 903 and 905; the simulation result display areas 911 and 912; and the medical care expense display areas 922 and 923.

The display condition setting area 901 includes “Attention-Focused Index Selection” column, “Display Unit Selection” column, and “Display Time Period Input” column. In “Attention-Focused Index Selection” column, whether a simulation result is displayed on the basis of the number of persons or on the basis of a cost (medical care expense) is selected. In “Display Unit Selection” column, it is selected whether the attention-focused index is displayed on the basis of the accumulated value of the attention-focused index or on the basis of the value of the attention-focused index for each year. A time period (in years) during which a simulation is executed is input in “Display Time Period Input” column.

In the target narrowing-down condition setting areas 902 and 904, conditions under which simulation target persons are determined are displayed. When “Condition Editing” buttons 906 and 908 are operated by a user, subscreens on which conditions for selecting simulation target persons are displayed, and the user can input conditions. The conditions for selecting simulation target persons are a parent population, the range of ages, the range of medical care expenses, and the like. In the intervention plan setting areas 903 and 905, intervention plans created in the intervention editing processing are displayed. When “Intervention Editing” buttons 907 and 909 are operated by a user, subscreens into which intervention plans are input are displayed, and the user can input intervention plans.

When “Simulation Executing” button 921 is operated by the user, the flow proceeds to step 802 of simulation execution processing, and the simulation executing unit 111 executes the simulation with the use of target narrowing-down conditions and intervention plans set by the user. After the simulation execution processing is finished, the results of the simulation are displayed in the simulation result display areas 911 and 912, and in the accumulated medical care expense display areas 922 and 923.

The way of transiting from one disease to others starting from a group of candidate diseases is displayed in the simulation result display areas 911 and 912. Each clinical condition is represented by a predefined figure (by a circle in the case of FIG. 9), and the size of the figure is decided according to the magnitude of the relevant attention-focused index (medical care expense or the number of persons) set in the display condition setting area 901 (for example, in proportion to the magnitude of the relevant attention-focused index). Connections from one node to another node is represented by an edge the presence of which is determined by the transition probability between the one node to the another node (for example, the edge is provided if the transition probability is larger than a predefined value, and the number of high transition probabilities is within a predefined number).

The results of the simulation are displayed in the simulation result display areas 911 and 912. To put it concretely, the simulation result display area 911 displays results, which are obtained by executing the simulation with conditions set in the target narrowing-down condition setting area 902 and the intervention plan setting area 903, with the use of a condition set in the display condition setting area 901. In addition, the simulation result display area 912 displays results, which are obtained by executing the simulation with conditions set in the target narrowing-down condition setting area 904 and the intervention plan setting area 905, with the use of a condition set in the display condition setting area 901. As mentioned above, displaying plural simulation results (for example, two) in parallel makes it possible to easily compare the transitions of the number of persons and medical care expenses as the predictions of plural intervention plan effects.

In each of the simulation result display areas 911 and 912, the medical care expense (or the number of persons) of each disease at a time point during a time period set as a display condition is displayed. A time point, at which the simulation results are displayed, is displayed on the upper right side of each of the simulation result display areas 911 and 912. States, which will be at the time point of the year 2020, are predicted and displayed in the case of FIG. 9.

Furthermore, in the simulation result display areas 911 and 912, simulation results during the time period set as the display condition can be dynamically displayed. In the case of FIG. 9, because a time period 5 years is set in the display condition setting area 901, a simulation is executed during a time period from the current time to the time five years ahead, and the simulation results are dynamically displayed at predefined intervals (for example, every year). In other words, because medical care expenses (or the number of persons) at the respective time points are different from each other, the sizes of the figures representing the respective clinical conditions changes dynamically. In this case, it is recommendable that small circles according to the number of persons who transit from one disease to another are displayed on an edge between nodes.

The accumulated medical care expense display area 922 displays accumulated medical care expenses for respective diseases of simulation 1 and those of simulation 2 distinctively with the use of bar graphs. The bar graphs displayed in the accumulated medical care expense display area 922 are displayed in conjunction with the contents displayed in the simulation result display areas 911 and 912 in terms of time. In other words, when the simulation result display areas 911 and 912 dynamically display the simulation results during the time period set as a display condition, the bar graphs displayed in the accumulated medical care expense display area 922 are dynamically displayed so that the bar graphs extend in synchronization with the contents displayed in the simulation result display areas 911 and 912. Simulating the transitions of the accumulated medical care expenses for the respective clinical conditions makes it possible to compare the effects of plural intervention plans with each other for the respective clinical conditions (for example, a case where an intervention menu is performed and a case where the intervention menu is not performed). In particular, it is possible to know on a medical care expense for which clinical condition a high reduction effect is exerted.

In addition, in the accumulated medical care expense display area 923, the transition of the accumulated medical care expense of all the diseases obtained by the simulation 1 and the transition of the accumulated medical care expense of all the diseases obtained by the simulation 2 are displayed by line graphs. The line graphs displayed in the accumulated medical care expense display area 923 are displayed in conjunction with the contents displayed in the simulation result display areas 911 and 912 in terms of time. In other words, when the simulation result display areas 911 and 912 dynamically display the simulation results during the time period set as a display condition, the line graphs displayed in the accumulated medical care expense display area 923 are dynamically displayed so that the line graphs extend in synchronization with the contents displayed in the simulation result display areas 911 and 912. Simulating the transition of the accumulated medical care expense of all the diseases makes it possible to compare the long-term effects of plural intervention plans on the entire medical care expense with each other (for example, a case where an intervention menu is not performed and a case where the intervention menu is performed). In particular, it is possible to know a time when the reduction amount of the medical care expense exceeds the introduction cost of the intervention plan, so that whether the cost of the intervention plan is recoverable or not can be judged.

In the above descriptions, although a system, in which the transition of a person's clinical condition is predicted, and the medical care expense of a group to which the person belongs and the like are simulated, has been explained, the present invention can also be applied to other variations. For example, a case where a medical institution introduces a new checkup apparatus or a new therapeutic instrument will be explained. If a new checkup apparatus or a new therapeutic instrument is introduced, transition probabilities among clinical conditions are changed because the accuracy of a checkup is improved, an early detection is realized, and a medical treatment that has not been given so far becomes usable. In this case, the receipts and disbursements of the medical institution are affected by the increase in the number of diseases that become treatable, the increase in the number of acceptable patients owing to the reduction of therapeutic periods (periods of hospitalization), the improvement in the operational efficiency of medical staffs, and the like. Modeling the above changes regarding the receipts and disbursements makes it possible to treat the introduction of the checkup apparatus and the therapeutic instrument similarly to the intervention effect models described in this embodiment. With this, the analysis system 100 of this embodiment can be utilized as a management simulation of a medical institution for simulating a problem how many years it takes to recover the introduction cost of the above-mentioned apparatus and instrument.

As described above, this embodiment according to the present invention includes: the model applying unit 113 that predicts at least one change among changes between the conditions of target persons in the case of an intervention not being followed and the conditions of the target persons in the case of the intervention being followed with reference to the health checkup information 121, the clinical condition transition model information 131, and the intervention effect model information 132; and the simulation executing unit 111 that predicts medical care expenses using the conditions predicted by the model applying unit 113, and calculates the medical care expense of a group to which the target persons belong by aggregating the predicted medical care expenses of the individual target persons. Because the simulation executing unit 111 outputs screen data used for displaying the calculated medical care expense of the group, calculating the effect for the group by accumulating the effects for the individual target persons makes it possible to select intervention plans suitable for the characteristics of the persons belonging to the group respectively instead of selecting intervention plans suitable for the effect on the whole group.

Furthermore, the model applying unit 113 predicts a first condition and a second condition that respectively correspond to intervention plans different from each other, and the simulation executing unit 111 predicts a first medical care expense and a second medical care expense using the first condition and the second condition respectively, aggregates the predicted first medical care expenses and the predicted second medical care expenses of the individual target persons, calculates the first medical care expense and the second medical care expense of the group to which the target persons belong respectively, and outputs screen data for displaying the first medical care expense and the second medical care expense in such a way that both expenses can be compared with each other. Therefore the predicted values of medical expenses calculated under plural conditions can be displayed in such a way that these values can be easily compared with each other.

In addition, the model applying unit 113 predicts the changes of the conditions of the individual target persons at predefined intervals during an input time period with reference to the health checkup information 121, the clinical condition transition models 131, and the intervention effect model information 132, the simulation executing unit 111 predicts the changes of the medical care expenses of the individual target persons at the predefined intervals during the input time period using the predicted conditions of the individual target persons, calculates the medical care expense of the group, to which the target persons belong, at the predefined intervals by aggregating the predicted medical care expenses at the predefined intervals, and outputs screen data for displaying the variation of the calculated medical care expense during the input time period. Therefore, the variation of the calculated medical care expense with time can be displayed in an easy-to-understand manner.

Furthermore, the simulation executing unit 111 outputs screen data for displaying a line graph showing the accumulated values of the calculated medical care expense of the group during the input time period. Therefore, it is possible to know a time when the reduction effect of the medical care expense exceeds the intervention cost.

In addition, intervention plans are regarded as plans for suppressing the medical care expenses of the target persons. Therefore, the reduction effect of the medical care expense of each plan can be learned.

Furthermore, the simulation executing unit 111 outputs screen data for displaying the result of the simulation with the use of a graphical model including edges that connect nodes with each other, wherein the conditions of the target persons are defined as the nodes respectively, and determines the magnitudes of the nodes in accordance with the amounts of expenses required under the conditions corresponding to the relevant nodes respectively. Therefore, the costs of the respective conditions can be displayed in an easy-to-understand manner.

Typical aspects according to the present invention other than the aspects that have been described in the appended claims can be cited as follows.

### Paragraph 1

An analysis system that including a processor and a memory connected with the processor,

the analysis system being capable of accessing a database that includes: health checkup information including the health checkup results of target persons; medical information including the medical care expenses of the target persons; and clinical condition transition models in which probabilistic dependencies between nodes corresponding to probability variables representing the conditions of the target persons and nodes corresponding to probability variables of factors that change the conditions are defined by directed edges or undirected edges,

wherein the analysis system further includes: a model applying unit in which the processor predicts at least one change among changes between the conditions of the target persons in the case of an intervention plan not being followed and the conditions of the target persons in the case of the intervention plan being followed with reference to the health checkup information, the medical information, and the clinical condition transition models; and

a simulation unit in which the processor predicts, with the use of the conditions predicted by the model applying unit, the number of persons under the condition, and

wherein the simulation unit outputs screen data used for displaying the calculated number of persons.

### Paragraph 2

The analysis system according to paragraph 1,

wherein the model applying unit predicts a first condition and a second condition that respectively correspond to intervention plans different from each other, and

the simulation unit predicts the first number of persons under the first condition and the second number of persons under the second condition, and

outputs screen data for displaying the first number of persons and the second number of persons in such a way that both numbers can be compared with each other.

### Paragraph 3

The analysis system according to paragraph 1,

wherein the model applying unit predicts the changes of the conditions of the individual target persons at predefined intervals during an input time period with reference to the health checkup information, the medical information, and the clinical condition transition models, and

the simulation unit predicts the changes of the number of persons under the respective conditions at the predefined intervals during the input time period using the predicted conditions of the individual target persons, and

outputs screen data for displaying the variation of the calculated medical care expense during the input time period.

### Paragraph 4

The analysis system according to paragraph 1,

wherein the intervention plan is a plan for suppressing the medical care expenses of the target persons.

### Paragraph 5

The analysis system according to paragraph 1,

wherein the simulation unit outputs screen data for displaying the result of the simulation with the use of a graphical model including edges that connect nodes with each other, wherein the conditions of the target persons are defined as the nodes respectively, and

determines the magnitudes of the nodes in accordance with the numbers of persons under the conditions corresponding to the nodes.

Here, the present invention is not limited to the above-described embodiments, and various modification examples and similar configurations can be included within the spirit and the scope of the appended claims. For example, the above embodiments are explained in detail for making the present invention easily understood, and therefore the present invention is not necessarily required to include all the configurations that have been described so far. In addition, a part of the configuration of a certain embodiment can be replaced with a part of the configuration of another embodiment. Furthermore, a part of the configuration of another embodiment can be added to a certain embodiment. In addition, a new embodiment of the present invention may be made by adding a different configuration to a part of the configuration of each embodiment, by deleting a part of the configuration from each embodiment, or by replacing a part of configuration of each embodiment with a different configuration.

Furthermore, some or all of the above configurations, functions, processing units, processing means, and the like can be realized by hardware, for example, by designing those using integrated circuits or realized by software through a processor's interpreting and executing programs that realize the respective functions.

Information regarding programs, tables, files, and the like, which realize the respective functions, can be recorded in memory devices such as a memory, and a hard disk, an SSD (Solid State Drive), or recording media such as an IC card, an SD card, and a DVD.

Furthermore, in the above-described drawings, control lines and information lines are shown in the case where they are indispensable for explaining the above embodiments, therefore all control lines and information lines required for implementing the above embodiments are not necessarily shown. It is conceivable that in reality almost all components in almost every embodiment are interconnected.

1. An analysis system comprising a processor and a memory connected with the processor,
the analysis system being capable of accessing a database that includes: health checkup information including the health checkup results of target persons; medical information including the medical care expenses of the target persons; and clinical condition transition models in which probabilistic dependencies between nodes corresponding to probability variables representing the conditions of the target persons and nodes corresponding to probability variables of factors that change the conditions are defined by directed edges or undirected edges,
wherein the analysis system further comprises: a model applying unit in which the processor predicts at least one change among changes between the conditions of the target persons in the case of an intervention not being followed and the conditions of the target persons in the case of the intervention being followed with reference to the health checkup information, the medical information, and the clinical condition transition models; and
a simulation unit in which the processor predicts medical care expenses using the conditions predicted by the model applying unit, and calculates the medical care expense of a group to which the target persons belong by aggregating the predicted medical care expenses of the individual target persons, and
wherein the simulation unit outputs screen data used for displaying the calculated medical care expense of the group.

2. The analysis system according to claim 1,
wherein the model applying unit predicts a first condition and a second condition that respectively correspond to intervention plans different from each other, and
the simulation unit predicts a first medical care expense and a second medical care expense for each of the target persons using the first condition and the second condition respectively,
aggregates the predicted first medical care expenses and the predicted second medical care expenses of the individual target persons respectively, and calculates the first medical care expense and the second medical care expense respectively of the group to which the target persons belong, and
outputs screen data for displaying the first medical care expense and the second medical care expense in such a way that both expenses can be compared with each other.

3. The analysis system according to claim 1,
wherein the model applying unit predicts the changes of the conditions of the individual target persons at predefined intervals during an input time period with reference to the health checkup information, the medical information, and the clinical condition transition models, and
the simulation unit predicts the changes of the medical care expenses of the individual target persons at the predefined intervals during the input time period using the predicted conditions of the individual target persons,
calculates the medical care expense of the group, to which the target persons belong, at the predefined intervals by aggregating the predicted medical care expenses of the target persons at the predefined intervals, and
outputs screen data for displaying the variation of the calculated medical care expense during the input time period.

4. The analysis system according to claim 3,
wherein the simulation unit outputs screen data for displaying a line graph showing the accumulated values of the calculated medical care expense of the group during the input time period.

5. The analysis system according to claim 1,
wherein the intervention is a plan for suppressing the medical care expenses of the target persons.

6. The analysis system according to claim 1,
wherein the simulation unit outputs screen data for displaying the result of the simulation with the use of a graphical model including edges that connect nodes with each other, wherein the conditions of the target persons are defined as the nodes respectively, and
determines the magnitudes of the nodes in accordance with the amounts of expenses required under the conditions corresponding to the relevant nodes respectively.

7. An analysis method executed at a system that evaluates a health guidance,
the system including a processor that executes a program and a memory that stores the program, and
the system being capable of accessing a database that includes: health checkup information including the health checkup results of target persons; medical information including the medical care expenses of the target persons; and clinical condition transition models in which probabilistic dependencies between nodes corresponding to probability variables representing the conditions of the target persons and nodes corresponding to probability variables of factors that change the conditions are defined by directed edges or undirected edges,
wherein the method comprises: a model applying step in which the processor predicts at least one change among changes between the conditions of the target persons in the case of an intervention not being followed and the conditions of the target persons in the case of the intervention being followed with reference to the health checkup information, the medical information, and the clinical condition transition models; and
a simulation step in which the processor predicts medical care expenses using the conditions predicted by the model applying unit, and calculates the medical care expense of a group to which the target persons belong by aggregating the predicted medical care expenses of the individual target persons, and
wherein, in the simulation step, screen data used for displaying the calculated medical care expense of the group is output.

8. The analysis method according to claim 7,
wherein, in the model applying step, a first condition and a second condition that respectively correspond to intervention plans different from each other are predicted, and
in the simulation step, a first medical care expense and a second medical care expense for each of the target persons are predicted using the first condition and the second condition respectively,
the predicted first medical care expenses and the predicted second medical care expenses of the individual target persons are aggregated respectively, and the first medical care expense and the second medical care expense of the group to which the target persons belong are calculated respectively, and
screen data for displaying the first medical care expense and the second medical care expense is output in such a way that both expenses can be compared with each other.

9. The analysis method according to claim 7,
wherein, in the model applying step, the changes of the conditions of the individual target persons are predicted at predefined intervals during an input time period with reference to the health checkup information, the medical information, and the clinical condition transition models, and
in the simulation step, the changes of the medical care expenses of the individual target persons are predicted at the predefined intervals during the time period using the predicted conditions of the individual target persons,
the medical care expense of the group, to which the target persons belong, is calculated at the predefined intervals by aggregating the predicted medical care expenses of the target persons at the predefined intervals, and
screen data for displaying the variation of the calculated medical care expense during the input time period is output.

10. The analysis method according to claim 9,
wherein, in the simulation step, screen data for displaying a line graph showing the accumulated values of the calculated medical care expense of the group during the input time period is output.

11. The analysis method according to claim 7,
wherein the intervention is a plan for suppressing the medical care expenses of the target persons.

12. The analysis method according to claim 7,
wherein, in the simulation step, screen data for displaying the result of the simulation with the use of a graphical model including edges that connect nodes with each other is output, wherein the conditions of the target persons are defined as the nodes respectively, and
the magnitudes of the nodes are determined in accordance with the amounts of expenses required under the conditions corresponding to the relevant nodes respectively.
